(19) United States
(12) Patent Application Publication
(10) Pub. No.: US 2024/0324908 A1
(43) Pub. Date: Oct. 3, 2024

(54) Systems and Methods for Remotely Assessing a Condition in a Patient

(71) Applicant: Modality.AI, San Francisco, CA (US)

(72) Inventors: Vikram Ramanarayanan, San Francisco, CA (US); Michael Neumann, Winterbach (DE); William Burke, Portland, OR (US); David Pautler, Sarasota, FL (US); Hardik Kothare, Burlingame, CA (US); Doug Habberstad, Savanna, GA (US); Oliver Roesler, Weyhe (DE); Jackson Liscombe, Mill River, MA (US); Andrew Cornish, Gore (NZ); David Suendermann-Oeft, San Francisco, CA (US)

(21) Appl. No.: 18/130,135

(22) Filed: Apr. 3, 2023

(51) Int. Cl. A61B 5/11; A61B 5/00; G16H 50/30; G16H 80/00

(52) U.S. Cl. CPC A61B 5/1124; A61B 5/7475; G16H 50/30; G16H 80/00

(57) ABSTRACT

A system for administering performance tests to patients remotely has a server that can execute one or more performance tests, presented to the patient via a virtual agent running on the patient's computing device. The performance tests ask the patient to perform a task and then the patient's computing device captures the performance of the task such that the server can analyze the results and determine whether the patient suffers from a condition. The system is capable of adapting based on how a patient performs the tests, whether additional tests are needed or due to technical difficulties.

### FIELD OF THE INVENTION

The field of the invention is remote or virtual diagnosis of patient conditions.

### BACKGROUND

The background description includes information that may be useful in understanding the present invention. It is not an admission that any of the information provided herein is prior art or relevant to the presently claimed invention, or that any publication specifically or implicitly referenced is prior art.

The advent of telemedicine made it possible for people to get more care with less travel. This is particularly beneficial for patients who suffer from conditions that make it difficult or impossible to move. With the ability to speak with their care provider, routine medical events such as checkups and minor condition change could safely and effectively handled remotely. Additionally, greater care could be provided to those living in remote areas.

However, even with the advances in remote patient interaction and assistance, problems remain. The ability to automate tests remains very limited. Existing systems are not capable of adapting to conditions in terms of providing and measuring the tests, and are unable to calibrate the tests to adapt to a condition that the patient is demonstrating in real time.

Additionally, current remote medicine technologies are incapable of adapting to challenges associated with the patient taking tests as well as the conditions surrounding them.

Thus, there is still a need for a system that can effectively and accurately administer performance tests remotely and that can account for the various possibilities associated with remote access.

### SUMMARY OF THE INVENTION

The inventive subject matter provides apparatus, systems and methods in which a virtual agent can administer a performance test to a patient. A sensor on a client's computing device captures sensor data (e.g., video data) of the patient performing the test. A computing device, possibly located remotely, analyzes the performance of the test based on the captured sensor data and determines an existence of a condition based on the analyzed performance information. Upon determining the existence of a condition, the system can route this information to an appropriate party such as a provider. The selection of the provider can be based on the detected condition itself.

In embodiments of the inventive subject matter, the performance test is a cognitive test such as one whereby the patient must sequentially touch their eyes, nose and chin. In these embodiments, the computing device compares one or more of an order of the steps against a baseline, a speed of execution of the steps against a baseline, an accuracy of contact against a baseline, and a fluidity of motion against a baseline and detects a failure or deficiency in the performance according to one or more of these metrics.

Depending on which of the metrics above are deficient, the computing device can determine whether a potential condition is a motor condition, a cognitive condition, or both.

In embodiments of the inventive subject matter, the system can obtain a second performance test based on one or more of the performance of the first test by the patient or a detected existing condition. The second test is administered by the virtual agent. The performance of the second test is captured by the sensor (e.g., video camera) and the performance is analyzed by the computing device, which ascertains a severity of the detected condition.

In embodiments of the inventive subject matter, the system can make adjustments based on the various components of the system failing to meet performance requirements for a particular test. The computing device can track and analyze system metrics and compare those against required metrics for a particular performance test. If the system metrics do not meet the required performance metrics, the computing device can, in embodiments, adjust the administering of the performance test by adjusting one or more aspects of the execution of the first performance test. For example, the computing device can transfer at least a portion of the performance test to the client's device so that the test is run locally rather than remotely.

In other embodiments, the computing device can select a second performance test that does meet the currently available system metrics and the virtual agent can instead administer the second performance test.

Various objects, features, aspects and advantages of the inventive subject matter will become more apparent from the following detailed description of preferred embodiments, along with the accompanying drawing figures in which like numerals represent like components.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a.” “an.” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### BRIEF DESCRIPTION OF THE DRAWING

FIG. 1 is a diagrammatic overview of a system according to embodiments of the inventive subject matter.

FIG. 2 is a flowchart with an overview of the administration of a performance test to a user by the components of the system, according to embodiments of the inventive subject matter.

FIG. 3 is a flowchart that provides additional details regarding the administration of the test at step 230 of FIG. 2, according to embodiments of the inventive subject matter.

### DETAILED DESCRIPTION

Throughout the following discussion, numerous references will be made regarding servers, services, interfaces, engines, modules, clients, peers, portals, platforms, or other systems formed from computing devices. It should be appreciated that the use of such terms, is deemed to represent one or more computing devices having at least one processor (e.g., ASIC, FPGA, DSP, x86, ARM, ColdFire, GPU, multi-core processors, etc.) programmed to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable media storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, the Internet, LAN, WAN, VPN, or other type of packet switched network.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

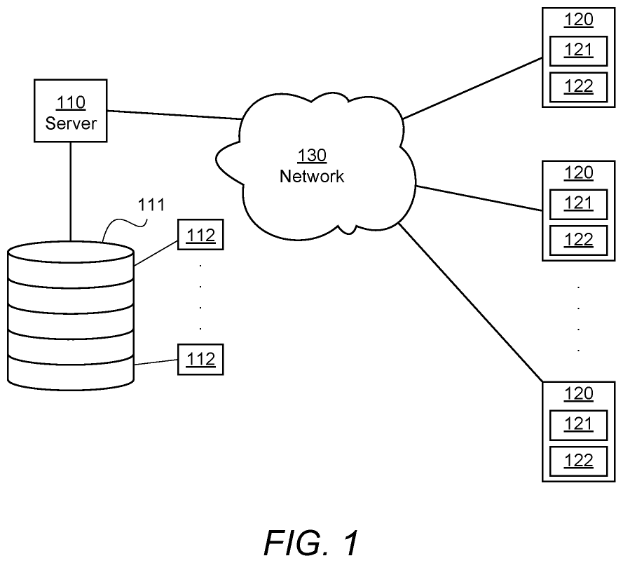

FIG. 1 is an illustrative diagram of the various components of system 100, according to embodiments of the inventive subject matter.

The system 100 includes a server 110 that can communicate to one or more client devices 120 over a network 130 (e.g., the internet). The server 110 can be one or more computing devices that include at least one processor, storage, and communication interface(s), located in one or more locations that can store and communicate data with other components of the system 100. The server 110 can include a database 111 that stores a plurality of performance tests 112.

The performance tests 112 include computer executable instructions that enable the system to administer a performance test to a user, obtain performance information captured via one or more sensors (e.g., a camera, microphone, etc.), and then enable the server 110 to analyze the performance of the test and determine whether a condition may exist.

Performance tests 112 in the database 111 can be associated with a particular type of test (e.g., mental, cognitive, emotional, motor skills). Additionally, the performance tests 112 can have an association with one or more conditions.

One example of a performance test 112 is a cognitive test whereby a patient is instructed to sequentially touch their eyes, nose and chin. This performance test is intended to test the patient's cognitive ability and assess the possibility of impairment. The test is scored based on the ability of the patient to identify each of the points of the face they are asked to touch and then on their ability to correctly do it in sequence. In this case, the possible condition determined is a level of impairment. If the patient is able to successfully perform the test by touching the points indicated and in the correct sequence, then this test shows no impairment. If the patient is able to identify one or more of the points, but either misses one or more points or performs it in a different sequence than the correct one, then the assessment is of “some impairment.” If the patient is unable to identify any of the points, then the assessment is “impairment.” In some variations of this test, a percentage of impairment can be provided based on the amount of the test that the patient performs correctly.

For the example performance test above 112, the database 111 stores the executable instructions that enables the presentation of instructions via the virtual agent 121 (which could be a video of someone performing the test), the capturing of the patient performing the test (such as via a video camera on the computing device 120), the analysis of the test to determine a condition (in this case, level of impairment) and the transmission of the test to appropriate parties (the patient themselves, health care providers, etc.).

In another example, a cognitive performance test may ask that the user recite as many animals starting with a certain letter in a certain amount of time. Based on their ability to recall animal names, the test can determine whether there is a certain amount of impairment. As with the example above, the database 111 stores the executable instructions that enable the system to present the performance test, capture the performance and evaluate the performance to determine whether any impairment exists.

The results of cognitive tests can lead to a determination (a diagnosis, a confirmation of, or an assessment of a likelihood of) of one or more degenerative cognitions such as Parkinson's, dementia, Alzheimer's, ALS, etc.

In embodiments of the inventive subject matter, the performance tests and the scoring thereof can include tests such as those included in the Montreal Cognitive Assessment and the Mini-Mental State Exam.

The client computing devices 120 can access the functions of the inventive subject matter via multiple ways. For example, a downloadable application or via a web portal accessible over a browser. The client computing devices 120 include at least one processor, at least one non-transitory computer-readable storage medium, and I/O interfaces that allow a user to receive data from and interact with the computing device 120 (e.g., monitor, touch screen, speakers, mouse, keyboard, cameras, etc.). The client computing devices 120 also have communication interfaces (e.g., Wi-Fi, wired internet connection, cellular, etc.) that enable the device 120 to exchange data over network 130. Examples of suitable computing devices 120 can include desktop computers, laptop computers, tablets, smartphones, and video game consoles.

To administer the tests and enable other interactions with a patient, a client computing device 120 executes a virtual agent 121. The virtual agent 121 can be installed on the client computing device 120. In other embodiments, the virtual agent 121 is executed by the server 110 and merely presented on the client computing device 120 via a web browser or other user-facing portal.

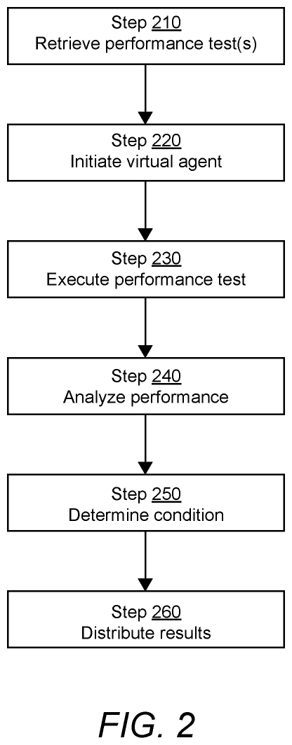

FIG. 2 is a flowchart with an overview of the administration of a performance test by the server 110 via the virtual agent 121 on the client computing device 120.

Prior to the process of FIG. 2., a user can access the functions of the system 100 by logging into their account via a web-browser or application installed on their device 120.

At step 210, the server 110 retrieves one or more performance tests that are to be presented to the user. The retrieval of the performance tests, including the selection of one or more of the performance tests, can be based on a previous recommendation or instruction, such as by the user's physician or other medical professional. For example, if a physician recommends performing tests to focus on motor skills/motor deterioration, the server 110 can select one or more performance tests designed to measure the user's motor skills and/or determine a condition of motor deterioration.

Other criteria for the selection of the performance test(s) can include age, gender, ethnic background, family history, medical history, profession, etc.

As an illustrative example, the server 110 selects the test discussed above whereby a user has to touch their eyes, nose and chin in sequence based on a request from a health care provider.

In embodiments of the inventive subject matter, the server 110 has a default base series of performance tests to administer that can cover multiple areas of a patient's well-being. For example, the base series can include one or more performance tests design to measure a person's cognitive ability, one or more tests designed to measure a person's motor skills, etc.

At step 220, the computing device 120 initiates the virtual agent 121. The initiation of the virtual agent 121 can be in response to the server 110 identifying that one or more performance tests need to be administered to this specific user. The virtual agent 121 can, in embodiments, be initialized based on a user logging on to their account and accessing a test.

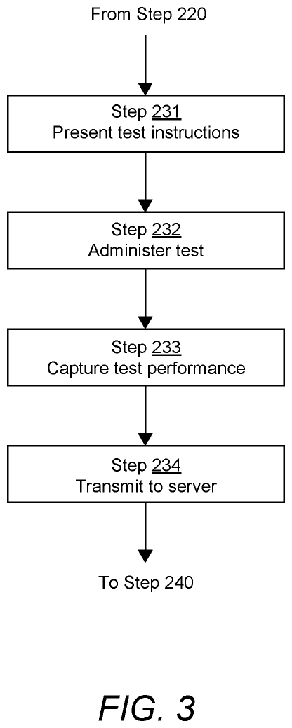

At step 230, the server 110 executes the test such that it is administered via the virtual agent 121. The detailed steps of the administration of the test can be seen in FIG. 3.

Via the virtual agent 121, the administration of the test can include presenting instructions for the test at step 231. This can include visual and/or audio instructions presented via the virtual agent 121 that explains the test to the user. The visual components of the instructions can include text, still images and/or video images.

Continuing with the example, the virtual agent 121 presents a video that shows the user the motions that are to be performed for the purposes of the performance test 112. In this example, the video would show a person sequentially touching their eyes, nose and chin in order. The video can also include audio instructions that describe the test as the video shows it to the user. The instructions can also include framing instructions such that the user is the right distance from the camera to enable the capture, as well as any additional instructions. The instructions can include a textual explanation of the test, along with a prompt to begin.

In another example, a performance test may ask a user to repeat a series of words. For this test, then, the virtual agent 121 can present the words audibly (via a speaker of computing device 120) and/or display them on the screen for a person to read. This can also include a prompt to begin reciting the words.

In another example, the performance test could be a test whereby a user is asked to name as many animals as they can in a certain time frame (e.g., 10 seconds). In this example, the administration of the test could be simply indicating, after the instructions of step 231, that the test will start and then starting a timer.

At step 232, the server 110 administers the performance test via the virtual agent 121. The administration of the performance test via the virtual agent 121 can vary depending on the test itself.

To begin the test, the virtual agent 121 can ask the user to click on a “ready” button or speak a word indicating they are ready. In embodiments, the virtual agent 121 can have a countdown or other indication that the test will begin shortly after the instructions of step 231 so that the user does not have to interact with the system at all to transition into the test.

At step 233, the computing device 120 captures the user's performance of the test 112 via one or more sensors 122 integral or connected to the computing device 120. Depending on the nature of the test, the sensors 122 could be one or more of a camera, a microphone, a touchscreen, a keyboard or mouse, etc.

For tests where a user is asked to perform a motion, the sensor 122 can be a camera or other device capable of capturing a user's motion during the performance of the test.

In the example of the “eyes, nose, chin” touch test, a camera 122 of the user's computing device 120 captures the user attempting to perform the steps of touching their eyes, nose and chin in sequence.

For another example, for tests where a user is asked to perform the test verbally (e.g., speaking certain words or phrases or reciting terms from memory), the sensor 122 could be a microphone or a camera with an incorporated microphone.

The sensor data captured by the sensor 122 during the performance of the test can be transmitted to the server 110 by the computing device 120 at step 234. The process then moves on to step 240.

At step 240, the server 110 analyzes the performance of the performance test based on the sensor data captured by sensor 122.

In embodiments, the analysis can be a comparison of the performance test against established metrics for that particular test, where the performance or non-performance of certain aspects of the test are marked or graded

To perform the analysis, the server 110 can employ image recognition techniques (for example, to be able to accurately determine the position of a user's hands relative to their face), speech recognition techniques (for example, to be able to determine the words that a person spoke and then determine aspects of the speech such as fluidity, volume, stutters, etc.), pattern recognition techniques and other techniques that make it possible for the server 110 to “understand” the performance of the test by the user.

Continuing with the illustrative example, the server 110 applies image recognition techniques to properly determine what areas of the face are the “eyes”, “nose” and “mouth” as well as the “hand” that moves relative to the face, and based on that determines whether a user touched their eyes, nose and chin based on the captured video, and determines whether it was done in sequence.

At step 250, the server 110 compares the performance of the test against one or more condition metrics that are representative of particular conditions. Generally speaking, the comparison can be a comparison of the performance of the test captured in the sensor data against a baseline or standard performance of the test.

The baseline can be derived from a collection of sensor data performed by a plurality of people that are considered “healthy” (i.e., not suffering from the condition to be tested).

The condition can be one or more of a degenerative condition, a neurological condition, a mental condition, an emotional condition, or a physical condition.

In the illustrative example, the server 110 determines how many of the eyes, nose and chin the user was able to touch and whether it was performed in the correct sequence. As discussed above, if the server 110 determines that the test was performed properly—each of the eyes, nose and chin were touched in the correct sequence—the server 110 assigns the condition of “unimpaired”. If the server 110 determines that some (but not all) of the touches were performed and/or were not performed in the correct sequence, the server 110 assigns the condition of “some impairment.” If the server 110 determines that none of the touches were correctly performed, the server 110 assigns the condition of “impairment.” As discussed above, the server 110 can in embodiments determine more nuanced conditions based on a percentage of the test performed properly and in sequence.

In embodiments of the inventive subject matter, the scoring algorithms used to score/measure the performance of the test by a user can be build using (logistic regression or random forest) classifiers, whereby the scoring algorithms use information such as the accuracy of saying a word, a percentage accuracy of touching a particular body part, etc. as input and outputting a score or decision. The training of the classifiers and additional details regarding a suitable example of this embodiment of the inventive subject matter can be found in the paper titled “Investigating the Utility of Multimodal Conversational Technology and Audiovisual Analytic Measures for the Assessment and Monitoring of Amyotrophic Lateral Sclerosis at Scale” by Neumann, et al published in Interspeech 2021, 30 Aug.-3 Sep. 2021., which is incorporated herein by reference.

At step 260, having determined a condition (and optionally, a severity), the server 110 can route the results to one or more recipients. One of the recipients can be the user themselves. Other recipients could be a health care provider, a pharmacy, etc.

In an example where a user is asked to name as many animals starting with a particular letter within a minute, and then the user does so at step 230, the server 110 can analyze the performance of the test at step 240 by first converting (via speech recognition techniques), speech to text and then comparing the text to an existing ontology/database of animal names and then at step 250 scores the results based on the matches. In this example, the analysis of step 240 can further include tracking the time it took to name the animals and the intelligibility/fluidity of the responses.

In another example, the performance test 112 is such that the server 110 (via virtual agent 121) recites a series of digits and asks the user to repeat them. Thus, the analysis at step 240 involves speech recognition of the digits spoke and a comparison against the digits provided by the virtual agent 121. At step 250, the server 110 scores the performance.

In embodiments, multiple tests (such as the examples given herein) can be administered in a single session and the results of each test can be combined by the server 110 into an overall score of cognitive functioning.

In embodiments of the inventive subject matter, the server 110 can be programmed to select a second performance test from the database 111 based on the results of a first performance test.

For example, if the user's performance for a particular performance test results in the determination of a condition at step 250, the server 110 can retrieve a performance test that is applicable to the condition itself (e.g., the potential for Alzheimer's) or to the type of condition (e.g., a neurological condition). The second performance test is then executed according to the steps of FIGS. 2-3. The result of the second performance test can serve to confirm the identification of an initial condition after the first performance test and/or determine a severity of the condition.

In embodiments of the inventive subject matter, the server 110 can be programmed to account for a poor performance on a test for reasons other than the test itself.

In these embodiments, the server 110 can be programmed to execute (via the virtual agent 121) a new performance test that is similar to the prior test. For example, if the analysis shows a poor performance for a spoken test, it may be that the poor performance is due to a person's knowledge rather than their mental capacity/acuity. This may result if the words a person is asked to recite are of a subject matter that the user is unfamiliar with, for example. If a person is asked to name cities starting with “S” but they are not knowledgeable about city names, then the result of the performance test is likely to be low but not in a way that reflects a condition related to mental acumen. Thus, the server 110 can retrieve a new test that asks the person to name animals with four legs, for example. If the results of the second test are still indicative of a condition, then that can be reported in step 260 above. In variations, additional tests can be selected and administered as discussed above. In a variation of this embodiment, the language of the performance test can be changed if English is not the user's primary language and then the same test administered in the user's primary language.

In a variation of these embodiments, a poor performance on a cognitive test based on a patient performing a motion or series of motions can be based on a difficulty for the user to move rather than a cognitive reason. For example, in the test that requires a person to touch, in sequence, their eyes, nose and chin, a person may accidentally touch their forehead, their check and their lips. In this case, it is clear that the person understood the test instructions and recalled the steps but a condition with regard to motor skills prevented their proper execution of the test. Using image recognition techniques the server 110 can recognize that the person attempted to perform the steps and in the correct order by determining that the placement of their hands was proximate (i.e., within a certain distance and far enough from the other parts) to the correct body part and in the correct order. In this embodiment, the server 110 can obtain a performance test 112 related to motor skills and dexterity and administer the test via the virtual agent 121 to determine whether a motor skills condition actually exists and if so, the severity thereof.

In embodiments of the inventive subject matter, the server 110 can adjust the administering of one or more performance tests based on conditions outside of the test itself. For example, to account for a drop or lack in a certain system resource, for a certain environmental condition etc.

In some of these embodiments, the server 110 can modify or otherwise adjust the administration of some or all of the performance test (e.g., one or more of the instructions, the administering of the test itself, etc.) based on a dip or unreliable network connection and/or a lack of sufficient computing resources of the computing device 120. In these embodiments, the performance tests can have one or more required resource metrics that are necessary for the test to be conducted normally. The resource metrics can include a metric for hardware requirements (e.g., a camera, microphone etc), a network bandwidth requirement (e.g., a minimum to be able to present video instructions in real-time or near-real-time and/or stream the results back from the computing device 120 in real-time or near-real-time), a computing hardware requirement (e.g., that the computing device 120 have certain capabilities to execute the virtual agent 121 and to present video, capture video at a certain resolution, etc.).

In these embodiments, the server 110 can perform checks to determine whether the resource metrics requirements for a test are met. This can include a check for the necessary hardware (e.g., a camera, microphone, etc.), a test of the available network bandwidth, and a test of the computing device 120 to determine the available resources.

If the metrics obtained by the server 110 do not meet the requirements for the performance test, the server 110 can adjust the administration of the test as follows:

In some embodiments of the inventive subject matter, the server 110 can cause the virtual agent 121 to change the modality of the administered test (e.g., from video to audio-only, from audio-only to text). For example, if some or all of the administration of the test is done over video, the virtual agent 121 can instead only present an audio instruction (thus saving the bandwidth of streaming video versus only audio).

In embodiments of the inventive subject matter, the server 110 can adjust by transferring some or all of the instructions (code) to administer the test from the server 110 to the computing device 120 such that the computing device 120 executes the performance test locally. In these embodiments, the computing device 120 can then store the results of the performance test locally until conditions improve and the computing device 120 can transmit the results to the server 110 for analysis. In a variation of these embodiments, the instructions transferred to the computing device 120 can include the instructions that enable the computing device 120 to execute some or all of the analysis.

In embodiments of the inventive subject matter, the server 110 can measure a delay (such as one caused by network lag) that affects the real-time or near-real time nature of the interaction between the virtual agent 121 and the user. The delay can be a delay between the executing of a part of the test process by the server 110 and the actual representation of that part by the virtual agent 121, a delay in a question and a response between the virtual agent 121 and the user, or a delay in capturing the user's performance of the test and relaying it in real-time to the server 110.

The server 110 can then account for the delay by buffering more of the test process on the client computing device 120, such as the video or instructions steps. The server 110 can also buffer the information captured via sensor 122 so that it is sent as an uninterrupted stream once the network conditions have improved. For tests that require a back-and-forth interaction between the server 110 (via virtual agent 121) and the user, the buffering can be so that the information presented is smooth and uninterrupted as so that the user can immediately respond in a natural manner.

In embodiments of the inventive subject matter, the server 110 can analyze one or more of the resource metrics against the required metrics for a particular performance test 112. Based upon one or more of the resource metrics not meeting the required metrics for the performance test 112, the server 110 can select a second performance test 112 where the resource metrics available meet the required metrics of the second performance test 112. The second performance test 112 will thus be a test that is used for a similar purpose of the first performance test 112 (such as to test a particular aspect of cognitive function, motor function, etc.), but that is less resource-intensive than the first test. For example, the second performance test 112 might only require the capture of audio instead of video and audio in the first test 112, thus having lower hardware requirements for the user computing device 120 and also for bandwidth requirements. The server 110 then obtains the second performance test 112 and proceeds to present it via the virtual agent 121 as discussed in the embodiments above.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method for remotely assessing the presence of a condition in patient, comprising:
administering, by a virtual agent and through a client computing device, a performance test;
capturing, by a sensor of the client computing device, sensor data of a patient performing the test;
analyzing, by a computing device, a performance of the performance test by the patient based on the sensor data;
determining, by the computing device, an existence of a condition based on the analyzed performance; and
routing, by the computing device, the determined existence of the condition to an appropriate provider based on the condition.

2. The method of claim 1, further comprising wherein:
the performance test comprises a three-step cognitive test whereby the client must sequentially touch their eyes, nose and chin;
the sensor is a camera;
the capturing step comprising capturing, by the camera, video data of the client performing the three-step test;
the analyzing step comprising analyzing, by the computing device, movements of the client captured in the video data against a baseline;
the determining step comprising determining, by the computing device, the existence of a degenerative condition based on the analysis of the movements of the client against the baseline.

3. The method of claim 2, wherein the analyzing step further comprises at least one of:
comparing, by the computing device, an order of the steps performed by the client against an order of the steps in the baseline;
comparing, by the computing device, a speed of execution of the steps against a speed in the baseline;
comparing, by the computing device, at least one contact region against at least one baseline contact region to determine an accuracy of contact;
comparing, by the computing device, a fluidity of motion of execution of the steps against a fluidity in the baseline; and
detecting, by the computing device, at least one of: a missed step in the performance of the test, an interruption in the fluidity of motion of the test, a low accuracy in the performance of the test; and a slow performance of the test.

4. The method of claim 3, further comprising:
determining that the condition is at least one of a motor condition or a cognitive condition based on the detected at least one of the missed step in the performance of the test, the interruption in the fluidity of motion of the test, the low accuracy in the performance of the test; and the slow performance of the test.

5. The method of claim 1, wherein the performance test comprises a first performance test from a plurality of performance tests to be administered the method further comprising:
obtaining, by the computing device and based on at least one of the existence of the condition and the performance of the performance test, a second performance test;
administering, via the virtual agent and through the client computing device, the second performance test;
capturing, by the sensor of the client computing device, a second sensor data of the patient performing the second performance test;
analyzing, by the computing device, a performance of the second performance test by the patient based on the second sensor data;
evaluating, by the computing device, a severity of the determined existence of the condition based on the analyzed performance of the second performance test.

6. The method of claim 1, further comprising:
analyzing, by the computing device, at least one system metric;
correlating, by the computing device, the at least one metric to a required metric of the first performance test;
determining that the at least one metric does not meet the required metric; and
adjusting the administration of the performance test by the virtual agent based on the failure to meet the required metric.

7. The method of claim 6, wherein the step of adjusting comprises at least one of:
changing a modality of presentation of the performance test;
transferring at least a portion of the performance test from a server to the client computing device; and
measuring a delay in at least one of the administering, capturing or analyzing steps and accounting for the delay in the analysis of the performance by the client.

8. The method of claim 6, wherein the performance test comprises a first performance test from a plurality of performance tests to be administered the method further comprising:
analyzing, by the computing device, at least one system metric;
correlating, by the computing device, the at least one metric to a required metric of the first performance test;
determining that the at least one metric does not meet the required metric; and
selecting a second performance test from the plurality of performance tests, the second performance test having a second required metric that is met by the at least one metric;
replacing the first performance test with the second performance test; and
administering, by the virtual agent and through the client computing device, the second performance test.
